(19) United States
(12) Patent Application Publication
(10) Pub. No.: US 2021/0290151 A1
(43) Pub. Date: Sep. 23, 2021

(54) PRESSURE ULCER PREVENTION

(71) Applicant: KONINKLIJKE PHILIPS N.V., ElNDHOVEN (NL)

(72) Inventors: Saman Parvaneh, Malden, MA (US); Richard Earl Gregg, Westford, MA (US); Saeed Babaeizadeh, Arlington, MA (US)

(21) Appl. No.: 16/326,982

(22) PCT Filed: Aug. 28, 2017
(86) PCT No.: PCT/EP2017/071485

§ 371 Date: Feb. 21, 2019

Related U.S. Application Data
(60) Provisional application No. 62/384,464, filed on Sep. 7, 2016.

(51) Int. Cl. A61B 5/00; A61B 5/318; A61B 5/389; A61B 5/11; A61G 7/057; A61G 9/00

(52) U.S. Cl. CPC A61B 5/445; A61B 5/7264; A61B 5/725; A61B 5/318; A61G 9/003; A61B 5/746; A61B 5/1116; A61G 7/05776; A61B 5/389

(57) ABSTRACT

Methods and systems for bedsore prevention. In accordance with various embodiments, methods and systems use information available from ECG and/or EMG sensor devices to determine whether a patient has performed a qualified movement within a predetermined time period. Upon determining a patient has not performed a qualified movement within the predetermined time period, a notification to that effect may be communicated to a clinical team member.

### TECHNICAL FIELD

Various embodiments described herein relate to methods and systems for preventing pressure ulcers and, more particularly but not exclusively, to methods and systems for preventing pressure ulcers using electrocardiogram and/or electromyography data.

### BACKGROUND

Pressure ulcers (commonly referred to as “bed sores”) are a common problem for patients in healthcare institutions such as hospitals or the like. Typically, patients develop pressure ulcers after sitting or lying in the same position for an extended period of time. Pressure ulcers can lead to further complications such as sepsis, localized infection, pain, morbidity, and mortality.

Pressure ulcers are also associated with high costs. In the U.S., for example, the prevalence of pressure ulcers in acute care settings ranges between 14% and 17%. The cost related to pressure ulcer prevention per patient per day varies between $17 and $98 across all types of health care settings. Pressure ulcer prevention plays an important role in improving patient care and reducing the cost of care.

One existing technique for preventing pressure ulcers is to follow a guideline prescribing when to physically turn a patient and to manually track how often the patient is turned. For example, clinical team members may turn a patient from lying on their back to lying on their side (and vice versa) every two hours and keep a log of dates and times each time the patient is turned. This technique, however, creates additional responsibilities and tasks for clinical teams and therefore may not be suitable for busy clinics/hospitals. Additionally, this technique may be inaccurate due to human errors.

Another existing technique for preventing pressure ulcers is to use a physiological monitoring device equipped with one or more accelerometers. However, many existing monitoring systems do not include accelerometers. Accordingly, this technique may be expensive and impractical as clinics/hospitals must modify or otherwise change their monitoring systems to include accelerometers.

Yet another existing technique for preventing pressure ulcers is for patients to use wearable devices (e.g., LEAF patient sensors) or to use mattresses that are specifically designed for preventing pressure ulcers. However, this can be impractical as it requires that new components be added to the clinic/hospital. Compatibility issues may also arise between these devices and existing medical systems.

A need exists, therefore, for methods and systems for pressure ulcer prevention that overcome these disadvantages.

### SUMMARY

This summary is provided to introduce a selection of concepts in a simplified form that are further described below in the Detailed Description section. This summary is not intended to identify key features or essential features of the claimed subject matter, nor is it intended to be used as an aid in determining the scope of the claimed subject matter.

In one aspect, various embodiments relate to a device for preventing a pressure ulcer. The device includes a communications interface for receiving at least one of electrocardiogram data regarding a patient and electromyography data regarding the patient; a memory; and a processor, the memory storing instructions for configuring the processor to: execute at least one classification module to analyze at least one of the electrocardiogram data and the electromyography data and to provide an output related to the data; and execute an inference engine to determine whether the patient has performed a qualified movement within a predetermined time period based on the output from the at least one classification module, wherein the qualified movement is a movement sufficient to prevent a pressure ulcer.

In one embodiment, the at least one classification module includes an automatic nervous system activity classifier that is configured to detect a change in patient heart rate from the electrocardiogram data.

In one embodiment, the at least one classification module includes a mechanical displacement classifier that is configured to detect at least one of a change in cardiac axis of the patient and baseline wander by low pass filtering the electrocardiogram data.

In one embodiment, the at least one classification module includes a muscle activity classifier that is configured to detect muscle activity by at least one of high pass filtering the electrocardiogram data and direct analysis of the electromyography data.

In one embodiment, the at least one classification module includes a motion artifact classifier that is configured to detect a motion artifact in at least one of electrocardiogram data and electromyogram data due to movement of at least one electrode operably connected to the patient.

In one embodiment, the output from the at least one classification module includes one or more of a binary value, a weighted value, and a vote, and is used by the inference engine to determine whether the patient has performed a qualified movement within the predetermined time period.

In one embodiment, the processor is further configured to at least one of activate a pressure relief mattress, activate a bed pad system, and issue an alert to at least one clinical team member upon the inference engine determining the patient has not performed a qualified movement within the predetermined time period.

In another aspect, various embodiments relate to a method of preventing a pressure ulcer. The method includes receiving, via a communication interface, at least one of electrocardiogram data regarding the patient and electromyography data regarding the patient from at least one physiological monitoring device; executing, via a processor, at least one classification module to analyze at least one of the electrocardiogram data and the electromyography data and to provide an output related to the data; and executing, via the processor, an inference engine to determine whether the patient has performed a qualified movement within a predetermined time period based on the output from the at least one classification module, wherein the qualified movement is a movement sufficient to prevent a pressure ulcer.

In one embodiment, the at least one classification module includes an automatic nervous system activity classifier that is configured to detect a change in patient heart rate from the electrocardiogram data.

In one embodiment, the at least one classification module includes a mechanical displacement classifier that is configured to detect at least one of a change in cardiac axis of the patient and baseline wander by low pass filtering the electrocardiogram data.

In one embodiment, the at least one classification module includes a muscle activity classifier that is configured to detect muscle activity by at least one of high pass filtering the electrocardiogram data and direct analysis of the electromyography data.

In one embodiment, the at least one classification module includes a motion artifact classifier that is configured to detect a motion artifact in at least one of electrocardiogram data and electromyogram data due to movement of at least one electrode operably connected to the patient.

In one embodiment, the output from the at least one classification module includes one or more of a binary value, a weighted value, and a vote, and is used by the inference engine to determine whether the patient has performed a qualified movement within the predetermined time period.

In one embodiment, the method further includes at least one of sending an activation command to a pressure relief mattress, sending an activation command to a bed pad system, and issuing an alert, via the processor, to at least one clinical team member upon the inference engine determining the patient has not performed a qualified movement within the predetermined time period.

In yet another aspect, various embodiments relate to a computer readable medium containing computer-executable instructions for performing a method for preventing a pressure ulcer. The computer readable medium includes computer-executable instructions for receiving, via a communication interface, at least one of electrocardiogram data regarding a patient and electromyography data regarding the patient from at least one physiological monitoring device; computer-executable instructions for executing, via a processor, at least one classification module to analyze at least one of the electrocardiogram data and the electromyography data and to provide an output related to the data; and computer-executable instructions for executing, via the processor, an inference engine to determine whether the patient has performed a qualified movement within a predetermined time period based on the output from the at least one classification module, wherein the qualified movement is a movement sufficient to prevent a pressure ulcer.

### BRIEF DESCRIPTION OF THE DRAWINGS

In order to better understand various example embodiments, reference is made to the accompanying drawings, wherein:

FIG. 1 illustrates a system for preventing a pressure ulcer in accordance with one embodiment;

FIG. 2 depicts a flowchart of an algorithm implemented by the classification module 116 of FIG. 1 in accordance with one embodiment;

FIG. 3 illustrates a patient lying in the supine position;

FIG. 4 illustrates the cardiac axis of the patient of FIG. 3;

FIG. 5 illustrates a patient lying on their side;

FIG. 6 illustrates the cardiac axis of the patient;

FIGS. 7A and 7B illustrate a patient using a bed support system and a pressure relief mattress, respectively, in accordance with one embodiment; and

FIG. 8 illustrates an example of a hardware device for implementing the systems and methods described herein in accordance with one embodiment.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment.

Some portions of the description that follow are presented in terms of symbolic representations of operations on non-transient signals stored within a computer memory. These descriptions and representations are used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each may be coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

The features described herein overcome the disadvantages of existing techniques by using readily available (and commonly used) equipment to gather information relevant to pressure ulcer prevention. More specifically, features described herein may use electrocardiogram data, electromyography data, or some combination thereof, to determine whether or not a patient has performed a qualified movement within a predetermined period of time.

In the context of the present application, the term “qualified movement” or “qualified body movement” may refer to any patient movement sufficient to prevent a pressure ulcer from developing. This may include movement performed by the patient themselves, or movement due to a clinical team member or other personnel physically moving the patient (e.g., moving the patient from lying on their back to lying on their side).

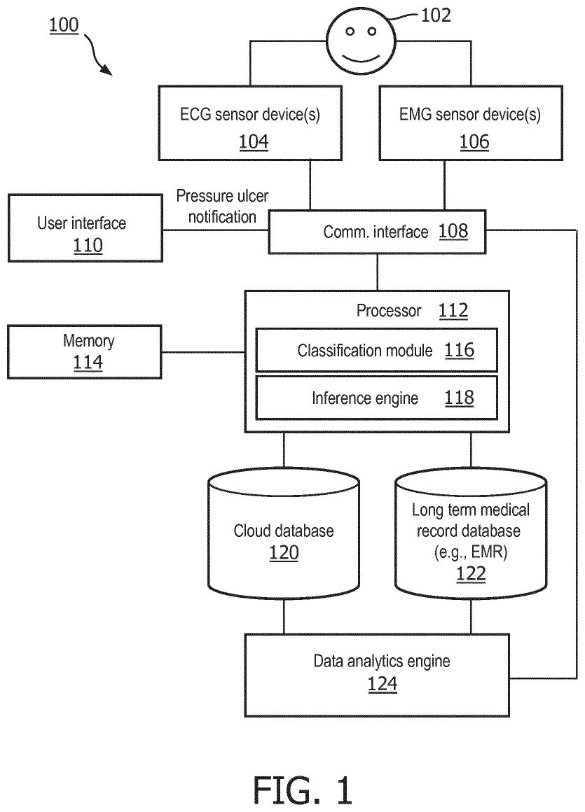

FIG. 1 illustrates a system 100 for preventing a pressure ulcer from developing on a patient 102 in accordance with one embodiment. This system 100 may be implemented in hospitals, nursing homes, patient residences, urgent care facilities, rehabilitation facilities, physician offices, or any other environment in which patients are at risk of developing pressure ulcers.

The patient 102 may be connected to various sensor devices as part of their medical monitoring and treatment plans. These may include electrocardiogram (ECG) sensor devices 104 and/or electromyography (EMG) sensor devices 106.

In accordance with standard ECG practice, the ECG sensor devices 104 may include a plurality of electrodes placed on the patient's skin at various locations. For example, a conventional 12-lead ECG includes electrodes placed on the patient's left arm, right arm, left foot, right foot, and several electrodes placed on the patient's chest.

The electromyography sensor devices 106 may include a plurality of sensor devices operably connected to a patient to gather data regarding the patient's muscle activity. These may include electrodes placed on the patient's skin or underneath the patient's skin (e.g., as a needle electrode inserted into a patient's muscles).

Data from the ECG sensor device(s) 104 and/or the EMG sensor device(s) 106 may be communicated to other devices of the system 100 via a communication interface 108. The communication interface 108 may be configured as an Ethernet communications interface for a local area network (LAN), an RS-485 communication interface, a general purpose interface bus communications interface, an RS-232 communications interface, or any other type of communications interface whether available now or invented hereafter as long as it can communicate data from the sensor devices 104 and/or 106 to other components of the system 100 such as one or more user interfaces 110.

The user interfaces 110 may present instructions and information regarding the patient 102 to one or more clinical team members. A user interface 110 may be implemented as, for example, a laptop, PC, tablet, mobile device, a monitor, a haptic-based communication mechanism, or the like.

Data regarding the patient 102 may be communicated to a processor 112. The processor 112 may be in operable connectivity with a memory 114 that stores instructions for various modules to be executed on the processor 112. For example, the processor 112 may include a classification module 116 and an inference engine 118 when executing the stored instructions.

The processor 112 may also reset/set a timer at the time of the most recent detected qualified movement. For example, if the processor 112 detects a qualified movement at 4:00 PM, the processor 112 may accordingly reset/set a timer at 4:00 PM. Accordingly, the processor 112, as well as clinical team members, can monitor how much time has passed since the most recent qualified movement.

If data is inconclusive, the processor 112 may nonetheless instruct the user interface 110 to issue an alert to a clinical team member. Therefore, a clinical team member may be inclined to check on the patient and physically turn the patient if necessary.

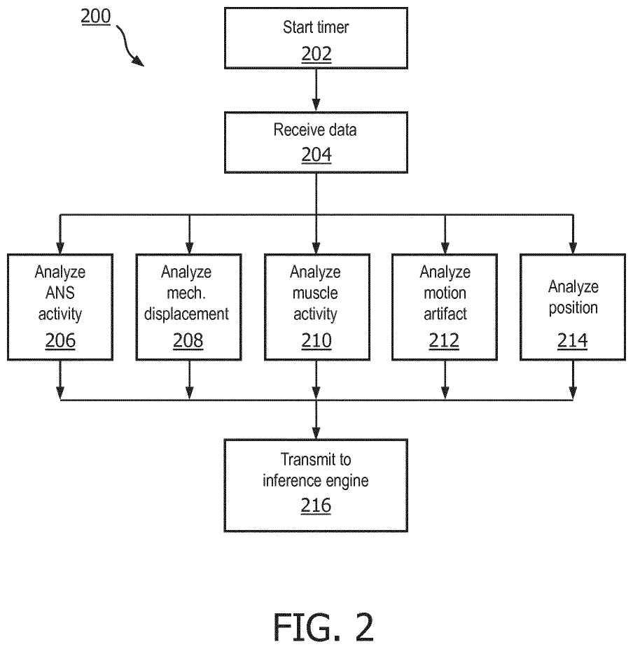

The classification module 116 may be any type of module that can analyze at least one of the electrocardiogram data and the electromyography data and provide an output related to the data. For example, the classification module 116 may execute the algorithm 200 shown in FIG. 2 to analyze the received data in a variety of ways. The classification module 116 may then provide an output based on the data to the inference engine 118.

The inference engine 118 may analyze the information from the classification module 116 in a variety of ways. In one embodiment, the classification module 116 may output binary values for each applicable analysis step of algorithm 200 (value 1: data suggests patient performed qualified movement; value 0: data suggests patient did not perform a qualified movement). For example, the classification module 116 may output a value of 1 for each of ANS activity, mechanical displacement, and muscle activity (if these types of data, discussed below, suggest the patient performed a qualified movement), and a value of 0 for each of motion artifact and position (if these types of data, discussed below, suggest the patient did not perform a qualified movement). The inference engine 118 may be configured to conclude the patient performed a qualified movement upon receiving two or more “yes” votes, for example.

The inference engine 118 may also consider various states of the patient 102 based on detected patterns in the data. For example, a relatively long period of low heart rate, followed by a brief spike in heart rate, followed by another relatively long period of low heart rate may suggest the patient performed a qualified body movement (as illustrated by the brief spike).

If the inference engine 118 determines that the patient has not performed a qualified body movement within a predetermined time period (e.g., within the previous two hours), a notification to that effect may be communicated to one or more clinical team members via one or more user interfaces 110. This notification may instruct the clinical team members to visit the particular patient and to physically turn the patient to prevent a pressure ulcer from developing. This notification may be presented via visual-based methods, audio-based methods, haptic-based methods, or some combination thereof.

If the inference engine 118 determines the patient 102 has performed a qualified movement within the predetermined time period (e.g., two hours), there may be no reason to issue an alert to a clinical team member because there is no immediate need to turn the patient. This inevitably saves time for clinical team members and allows them to focus on other tasks. However, the user interface 110 may nonetheless inform clinical team members if and when a patient performed a qualified movement if desired.

The inference engine 118 may require a higher probability that a high risk patient performed a qualified movement before, for example, resetting a timer. In other words, the inference engine 118 may be configured to analyze the obtained ECG/EMG data with more scrutiny before determining that the ECG/EMG data represents a qualified movement. For example, in the embodiment in which the classification module 116 outputs binary votes for each type of data analyzed in the algorithm 200 of FIG. 2, the inference engine 118 may require five “yes” votes before concluding the patient performed a qualified movement (rather than two votes).

FIG. 2 is a flowchart presenting one example of an algorithm 200 that may be executed by, e.g., classification module 116. Step 202 of the algorithm 200 involves starting a timer 202 to record the time lapsed since a certain start time. For example, this timer may be used to keep track of how long it has been since a patient last performed a qualified movement (or since a clinical team member moved the patient in a way that constitutes a qualified body movement).

Step 204 involves receiving data 204. This data may include data from the ECG sensors 104, the EMG sensors 106, or both. By using data from these readily available types of sensor devices, features described herein can glean information regarding the patient's movement (or lack thereof). Therefore, there is no need to equip a patient room or bed with additional types of sensor devices.

Step 206 of the algorithm 200 may involve analyzing automatic nervous system (ANS) activity obtained from ECG data. ANS activity may refer to changes in heart rate (exceeding a threshold, for example) that results from certain body movements. Accordingly, sudden changes in heart rate can be used to detect body movement. For example, an increase in heart rate occurring in a relatively short period of time may indicate that a patient has moved a certain amount (and possibly performed a qualified movement).

Step 208 of the algorithm 200 may involve analyzing the mechanical displacement 208 of the patient. As the patient moves (e.g., from a supine position to their side or vice versa), the relative location of the patient's heart with respect to electrodes of the electrocardiogram may change. As a result, the cardiac axis (i.e., the average direction of the flow of electricity through the patient's heart) may undergo a temporary change.

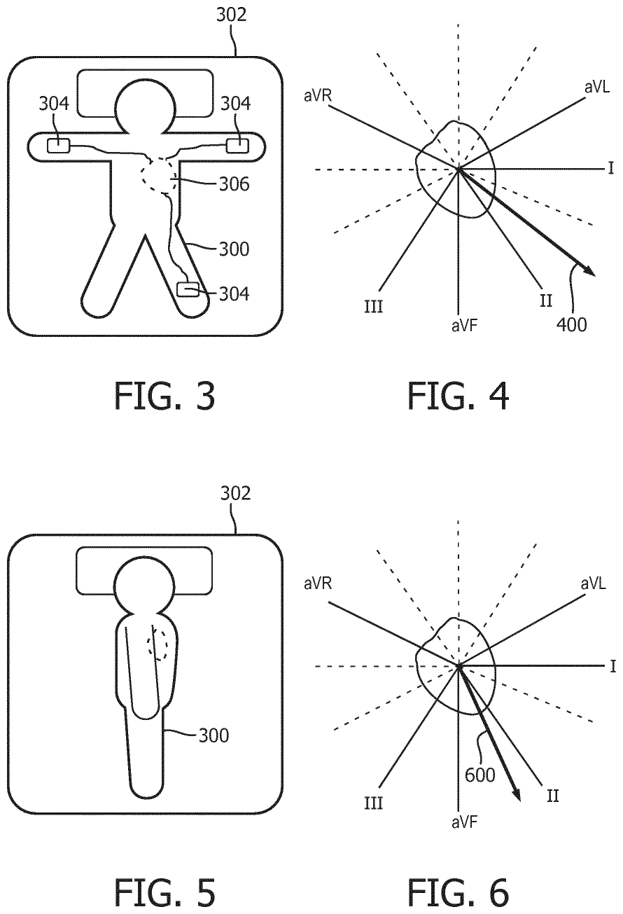

For example, FIG. 3 illustrates a patient 300 lying in the supine position on a bed 302. The patient 300 may be in a hospital or other type of healthcare institution. Also shown in FIG. 3 are ECG leads (sensors) 304 placed at various positions on the patient 300 to gather data regarding the patient's heart 306. Although a typical 12-lead electrocardiogram uses 6 additional sensors on a patient's chest and another sensor on the patient's right foot, these sensors are not shown in FIG. 3 for simplicity.

FIG. 4 depicts the cardiac axis 400 of the patient 300 when the patient 300 is in the supine position as in FIG. 3. Values regarding the cardiac axis 400 may be gathered, stored, and presented to a clinical team member via the user interface 110.

FIG. 5 illustrates the patient 300 of FIG. 3. However, the patient 300 is now lying on their side, as opposed to lying in the supine position as in FIG. 3. Although not shown, the ECG leads 304 of FIG. 3 are still monitoring the patient 300.

FIG. 6 depicts the cardiac axis 600 of the patient 300 when lying in the side position as in FIG. 5. As shown, the cardiac axis 600 may shift (albeit, only temporary) as a result of the patient 300 moving from the supine position to the side position. Accordingly, body movement can be detected based on mechanical displacement of the patient's heart by calculating the cardiac axis and tracking its changes. For example, changes in cardiac axis above a certain threshold (e.g., in terms of degrees) and/or changes that occur within a short time interval may indicate a qualified movement.

Body movement may also lead to displacement of electrodes on the patient's skin. This displacement may in turn lead to baseline wander in the ECG signals. However, baseline wander is a low frequency component in the ECG signal and can be eliminated by low pass filtering the ECG signal and the filtered signal used to detect a qualified movement.

Referring back to the algorithm 200 of FIG. 2, step 210 may involve analyzing muscle activity of the patient. For example, body movement (such as from moving from the supine position to side position and vice versa) activates certain muscles. This muscle activity may be captured by the ECG sensors 104 and identified by applying a high pass filter to the obtained ECG data.

In addition to using ECG data, step 210 may also be performed using the EMG sensor devices 106. EMG sensor devices 106 may be implemented as electrodes attached to the patient's skin (surface electrodes). Additionally or alternatively, the EMG sensor devices 106 may include one or more needle electrodes that are inserted into the patient's muscle (intramuscular electrodes).

Regardless of the exact configuration, the EMG sensor devices 106 may gather data regarding muscle activity that may be indicative of certain patient movements. For example, EMG sensor devices 106 may be operably connected to muscles that are generally used when a patient changes positions in a bed or in a chair (e.g., leg or arm muscles). Accordingly, certain muscle activity within a certain period of time may be indicative of a qualified movement.

Step 212 of algorithm 200 may involve analyzing artifacts due to motion. For example, patient movement may cause artifacts in ECG data by stretching the skin under one or more ECG electrodes. Additionally, patient movement may also cause wires that connect the ECG electrodes to a monitoring device to move, which may also create artifacts. Accordingly, these types of movements may create noise in the ECG (and/or the EMG) data. However, ECG noise level due to this motion artifact can be measured by a noise estimation algorithm and used to detect a qualified movement.

Algorithm 200, in certain embodiments, may also analyze information relating to the patient's position in step 214. For example, the processor 112 may be trained to associate certain ECG/EMG patterns with certain body positions. That is, a patient (or a large sample of patients) may generally output a certain ECG/EMG pattern when sitting, but output a different ECG/EMG pattern when lying in the prone position. Accordingly, the patient's position may be determined (or at least estimated) by their ECG/EMG data.

Information regarding associations between ECG data and positions may be learned using a supervised learning approach that considers data from one or many patients. This training method may involve having patients assume a plurality of different positions (sitting, standing, lying in the supine position, lying on their side, etc.) and recording the outputted data ECG/EMG data for each position. This information may be stored and used in conjunction with a variety of machine learning techniques such as a Hidden Markov Model to estimate the patient's position based on their ECG/EMG data. Accordingly, the processor 112 may recognize changes in patient position that may indicate qualified movements.

Steps 206-214 may be performed by one or more modules. For example, there may be different modules for each step (i.e., an ANS activity module to analyze ANS activity, a mechanical displacement module to analyze mechanical displacement, etc.) or a single module may implement one or more analytic techniques. For a given data set, another module (not shown) may calculate various derivatives of the data, including but not limited to simple averages (i.e., a mean(s), weighted averages, standard deviations, etc.).

The inference engine 118 may fuse the information obtained in steps 206-214 of algorithm 200 to determine whether the patient has performed a qualified movement within a predetermined time period. Not all information analyzed in FIG. 2 is required. For example, some embodiments may consider only ANS activity and mechanical displacement.

In one embodiment, the inference engine 118 may assign weights to each input feature obtained from any of the steps 206-214. For example, if the analysis of the mechanical displacement in step 208 indicates a high amount of changes in cardiac axis and/or significant magnitudes of changes in cardiac axis, the inference engine 118 may determine the patient has performed a qualified movement even if the other types of data suggest the patient did not perform a qualified movement.

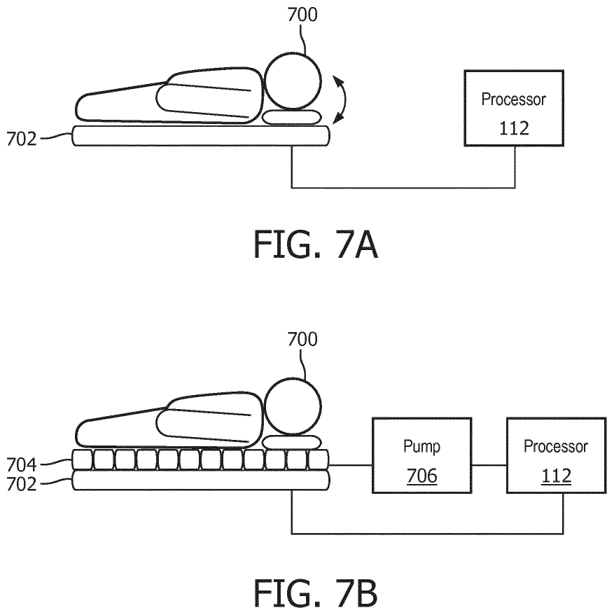

In addition to sending alerts, the processor 112 may also be in communication with, and activate, certain devices to affect the patient's position, orientation, and overall comfort. FIG. 7A, for example, illustrates a patient 700 lying on a bed pad 702. The bed pad 702 may be configured to incline/recline to change the position of the patient upon activation by the processor 112. For example, if the patient has not performed a qualified movement for a certain period of time, the processor 112 may activate the bed pad 702 to at least put the patient 700 in a different orientation.

Similarly, FIG. 7B illustrates the patient 700 lying on a pressure relief mattress 704 comprising a plurality of air bladders that may be inflated/deflated by a pump 706 and series of valves (not shown). Upon activation by the processor 112, the pump 706 may inflate certain air bladders of the pressure relief mattress 704 to change the pressure exerted on the patient 700.

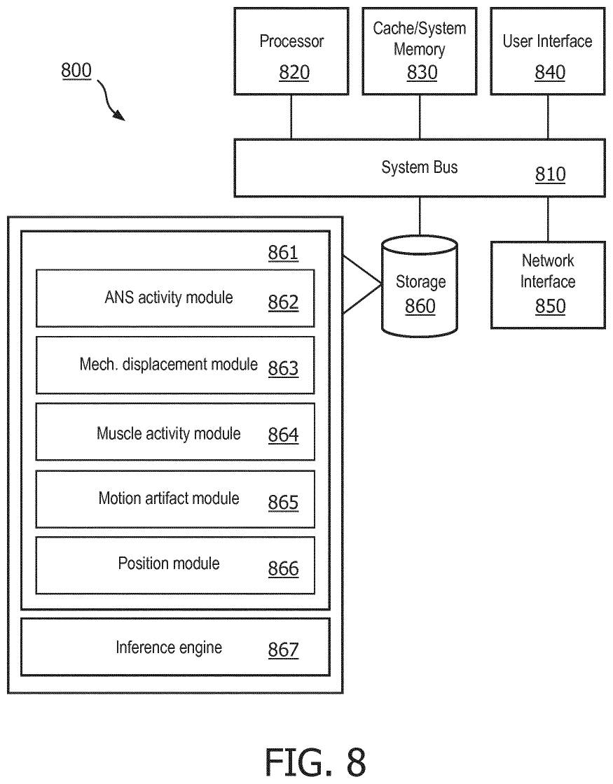

FIG. 8 illustrates an exemplary hardware device 800 for wirelessly transmitting data as described herein. As shown, the device 800 includes a processor 820, memory 830, user interface 840, network interface 850, and storage 860 interconnected via one or more system buses 810. It will be understood that FIG. 8 constitutes, in some respects, an abstraction and that the actual organization of the components of the device 800 may be more complex than illustrated.

The processor 820 may be any hardware device capable of executing instructions stored in memory 830 or storage 860 or otherwise capable of processing data. As such, the processor may include a microprocessor, field programmable gate array (FPGA), application-specific integrated circuit (ASIC), or other similar devices.

The memory 830 may include various memories such as, for example L1, L2, or L3 cache or system memory. As such, the memory 830 may include static random access memory (SRAM), dynamic RAM (DRAM), flash memory, read only memory (ROM), or other similar memory devices.

The user interface 840 may include one or more devices for enabling communication with a user. For example, the user interface 840 may include a display, a mouse, and a keyboard for receiving user commands In some embodiments, the user interface 840 may include a command line interface or graphical user interface that may be presented to a remote terminal via the network interface 850.

The network interface 850 may include one or more devices for enabling communication with other hardware devices. For example, the network interface 850 may include a network interface card (NIC) configured to communicate according to the Ethernet protocol. Additionally, the network interface 850 may implement a TCP/IP stack for communication according to the TCP/IP protocols. Various alternative or additional hardware or configurations for the network interface 850 will be apparent.

The storage 860 may include one or more machine-readable storage media such as read-only memory (ROM), random-access memory (RAM), magnetic disk storage media, optical storage media, flash-memory devices, or similar storage media. In various embodiments, the storage 860 may store instructions for execution by the processor 820 or data upon with the processor 820 may operate.

For example the storage 860 may include the classification module 861 that includes an ANS activity module 862 for analyzing ANS activity, a mechanical displacement module 863 for analyzing mechanical displacement, a muscle activity module 864 for analyzing muscle activity, a motion artifact module 865 for analyzing motion artifact, and a position module 866 for analyzing patient position. The storage 861 may further include the inference engine 867 to fuse the information from the various modules 862, 863, 864, 865, and 866 to determine whether or not a patient has performed a qualified movement within a predetermined period of time.

It will be apparent that various information described as stored in the storage 860 may be additionally or alternatively stored in the memory 830. In this respect, the memory 830 may also be considered to constitute a “storage device” and the storage 860 may be considered a “memory.” Various other arrangements will be apparent. Further, the memory 830 and storage 860 may both be considered to be “non-transitory machine-readable media.” As used herein, the term “non-transitory” will be understood to exclude transitory signals but to include all forms of storage, including both volatile and non-volatile memories.

While the device 800 is shown as including one of each described component, the various components may be duplicated in various embodiments. For example, the processor 820 may include multiple microprocessors that are configured to independently execute the methods described herein or are configured to perform steps or subroutines of the methods described herein such that the multiple processors cooperate to achieve the functionality described herein. Further, where the device 800 is implemented in a cloud computing system, the various hardware components may belong to separate physical systems. For example, the processor 620 may include a first processor in a first server and a second processor in a second server.

It should be apparent from the foregoing description that various example embodiments may be implemented in hardware or firmware. Furthermore, various exemplary embodiments may be implemented as instructions stored on a machine-readable storage medium, which may be read and executed by at least one processor to perform the operations described in detail herein. A machine-readable storage medium may include any mechanism for storing information in a form readable by a machine, such as a personal or laptop computer, a server, or other computing device. Thus, a machine-readable storage medium may include read-only memory (ROM), random-access memory (RAM), magnetic disk storage media, optical storage media, flash-memory devices, and similar storage media.

It should be appreciated by those skilled in the art that any block diagrams herein represent conceptual views of illustrative circuitry embodying the principles described herein. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention.

1. A device for preventing a pressure ulcer, the device comprising:
a communications interface for receiving at least one of electrocardiogram data regarding a patient and electromyography data regarding the patient;
a memory; and
a processor, the memory storing instructions for configuring the processor to:
execute at least one classification module to analyze at least one of the electrocardiogram data and the electromyography data and to provide an output related to the data; and
execute an inference engine to determine whether the patient has performed a qualified movement within a predetermined time period based on the output from the at least one classification module, wherein the qualified movement is a movement sufficient to prevent a pressure ulcer.

2. The device of claim 1, wherein the at least one classification module includes an automatic nervous system activity classifier that is configured to detect a change in patient heart rate from the electrocardiogram data.

3. The device of claim 1, wherein the at least one classification module includes a mechanical displacement classifier that is configured to detect at least one of a change in cardiac axis of the patient and baseline wander by low pass filtering the electrocardiogram data.

4. The device of claim 1, wherein the at least one classification module includes a muscle activity classifier that is configured to detect muscle activity by at least one of high pass filtering the electrocardiogram data and direct analysis of the electromyography data.

5. The device of claim 1, wherein the at least one classification module includes a motion artifact classifier that is configured to detect a motion artifact in at least one of electrocardiogram data and electromyogram data due to movement of at least one electrode operably connected to the patient.

6. The device of claim 1, wherein the output from the at least one classification module includes one or more of a binary value, a weighted value, and a vote, and is used by the inference engine to determine whether the patient has performed a qualified movement within the predetermined time period.

7. The device of claim 1, wherein the processor is further configured to at least one of activate a pressure relief mattress, activate a bed pad system, and issue an alert to at least one clinical team member upon the inference engine determining the patient has not performed a qualified movement within the predetermined time period.

8. A method of preventing a pressure ulcer, the method comprising:
receiving, via a communication interface, at least one of electrocardiogram data regarding a patient and electromyography data regarding the patient from at least one physiological monitoring device;
executing, via a processor, at least one classification module to analyze at least one of the electrocardiogram data and the electromyography data and to provide an output related to the data; and
executing, via the processor, an inference engine to determine whether the patient has performed a qualified movement within a predetermined time period based on the output from the at least one classification module, wherein the qualified movement is a movement sufficient to prevent a pressure ulcer.

9. The method of claim 8, wherein the at least one classification module includes an automatic nervous system activity classifier that is configured to detect a change in patient heart rate from the electrocardiogram data.

10. The method of claim 8, wherein the at least one classification module includes a mechanical displacement classifier that is configured to detect at least one of a change in cardiac axis of the patient and baseline wander by low pass filtering the electrocardiogram data.

11. The method of claim 8, wherein the at least one classification module includes a muscle activity classifier that is configured to detect muscle activity by at least one of high pass filtering the electrocardiogram data and direct analysis of the electromyography data.

12. The method of claim 8, wherein the at least one classification module includes a motion artifact classifier that is configured to detect a motion artifact in at least one of electrocardiogram data and electromyogram data due to movement of at least one electrode operably connected to the patient.

13. The method of claim 8, wherein the output from the at least one classification module includes one or more of a binary value, a weighted value, and a vote, and is used by the inference engine to determine whether the patient has performed a qualified movement within the predetermined time period.

14. The method of claim 8, further comprising at least one of sending an activation command to a pressure relief mattress, sending an activation command to a bed pad system, and issuing an alert, via the processor, to at least one clinical team member upon the inference engine determining the patient has not performed a qualified movement within the predetermined time period.

15. A computer readable medium containing computer-executable instructions for performing a method for preventing a pressure ulcer, the medium comprising:
computer-executable instructions for receiving, via a communication interface, at least one of electrocardiogram data regarding a patient and electromyography data regarding the patient from at least one physiological monitoring device;
computer-executable instructions for executing, via a processor, at least one classification module to analyze at least one of the electrocardiogram data and the electromyography data and to provide an output related to the data; and
computer-executable instructions for executing, via the processor, an inference engine to determine whether the patient has performed a qualified movement within a predetermined time period based on the output from the at least one classification module, wherein the qualified movement is a movement sufficient to prevent a pressure ulcer.
